(19) United States
(12) Patent Application Publication
(10) Pub. No.: US 2024/0369488 A1
(43) Pub. Date: Nov. 7, 2024

(54) ILLUMINATING TISSUE SAMPLES IN WIDE FIELD FLUORESCENCE LIFETIME IMAGING MICROSCOPY

(71) Applicant: Caelum Diagnostic Solutions, Inc., Venice, CA (US)

(72) Inventors: Lu Wang, San Jose, CA (US); Rachel Beth Wellner MD, Venice, CA (US); Derek S. Olender, San Jose, CA (US)

(21) Appl. No.: 18/637,120

(22) Filed: Apr. 16, 2024

Related U.S. Application Data
(60) Provisional application No. 63/500,595, filed on May 6, 2023.

(51) Int. Cl. G01N 21/64; G01N 33/483

(52) U.S. Cl. CPC G01N 21/6458; G01N 21/6408; G01N 21/6428; G01N 33/4833; G01N 2021/6439

(57) ABSTRACT

A method of evenly illuminating a tissue sample in a wide field Fluorescence Lifetime Imaging Microscopy (FLIM) and a system for practice thereof. Particularly provided here is a method of evenly illuminating a slide containing a tissue sample in a wide field light source by (i) illuminating the slide containing the tissue sample from more than one side, or (ii) illuminating the slide containing the tissue sample using a square wave excitation, or (iii) illuminating the slide containing the tissue sample using a combination of (i) and (ii) and capturing an individual wide field image or a plurality of wide field images of the tissue sample for accurate medical diagnosis of a medical condition.

### CLAIM OF PRIORITY

This application claims the benefit of priority of U.S. Provisional Patent Application Ser. No. 63/500,595, filed on May 6, 2023, the benefit of priority of which is claimed hereby, and which is incorporated by reference herein in its entirety.

### FIELD OF INVENTION

The invention generally relates to a method of illuminating samples in fluorescence lifetime imaging microscopy (FLIM).

### BACKGROUND

Tissue Biopsies are the current standard of care for the definitive diagnosis of cancer. Biopsy to collect tissue samples, followed by histopathology analysis of the samples to identify changes in tissues caused by disease, is critical to diagnose malignancy and determine patients' course of treatment. Despite being universally accepted as the standard of care, the current biopsy and pathology process is prone to several critical limitations that can adversely impact patient care and outcomes. This interval between the biopsy procedure and determining the findings has been associated with several undesirable effects, including patient injury, increased cost of care, and poor-quality outcomes.

The effect on clinical outcomes caused by the time required for biopsy results is manifested through the overall results of treatment. Patient care cannot commence until a diagnosis has been rendered. Studies have demonstrated that time to start treatment has a direct impact on the quality of outcomes for cancer patients. Even short delays to the start of treatment due to the time required for histology and pathology can impact a patient's individual response to treatment, and when looked at from a population health perspective, even relatively small delays to care can adversely impact survival rates.

There are multiple fluorescence imaging techniques and devices using one or the other features to measure the fluorescence spectrum formed by measuring the intensity of fluorescence used for sample imaging and diagnosis. Fluorescence Lifetime Imaging Microscopy (FLIM), however, uses a key property of fluorescence, which is lifetime, to analyze a sample. FLIM captures a fluorescence lifetime which reflects how long the dye stays in the excited state before returning to the ground state by emitting a fluorescence photon. The emission of a fluorescence photon from a fluorophore does not, however, always occurs at an exact time after excitation and a distribution of time is observed, showing an exponential decay function.

FLIM is generally based on two types of microscopies, namely scanning microscopy and wide field microscopy. In scanning microscopy, the fluorescence lifetime and fluorophore concentration are measured on a pixel-by-pixel base. At any given time, only one pixel is illuminated, and the fluorescence of that given pixel is then measured over time. Once the measurement for that pixel is completed, the next pixel is illuminated, and the cycle repeats. In wide field microscopy, the entire field is uniformly illuminated at once, and a special high-speed camera captures the changes in fluorescence over time.

FLIM can be performed in the time domain and in the frequency domain. In the time domain method, the fluorescence decay can be measured using time-coordinated single photon counting. Such measurements require short excitation pulses of high intensity and fast detection circuits. With the time domain techniques, lifetime is described from exponential fits to the fluorescence intensity data from a series of time windows. On the other hand, FLIM can also be performed in the frequency domain, where the excitation light intensity is programmably modulated at a certain frequency. The resulting fluorescence intensity is also a sine wave function. By comparing the phases and amplitudes of the excitation and fluorescence emission waveforms, the lifetime of fluorescence and concentration of fluorophores can be calculated.

Wide field FLIM, however, whether in the time domain or frequency domain, suffers from depth penetration restrictions impeding wider clinical applications such as using FLIM for cancer diagnosis in biological samples.

A biopsy tissue sample is approximately 1.6 mm in thickness, and the penetration depth of some excitation wavelengths is only on the order of tens of microns, leading to an issue when the cancerous tissue is located in the deeper tissue layers, there is a high probability that such tissue may not be visible for the FLIM device, thus providing false results and misdiagnosis. The excitation signal, as used in traditional FLIM devices, is heavily attenuated, and thus, only a small percentage of excitation photons may reach the deeper tissue or cell layers in the middle of the tissue sample, emitting a small number of photons and among these small numbers fewer will reach to the photon detection device.

Therefore, there still remain technical challenges to using FLIM technologies due to their low fluorescence intensity and poor tissue penetration under certain excitation frequencies. There is a need for enhanced technical solutions to solve the problem.

### SUMMARY

In summary, provided herein is a method of evenly illuminating a tissue sample in a wide field Fluorescence Lifetime Imaging Microscopy (FLIM) and a system for practice thereof. Particularly provided here is a method of evenly illuminating a slide containing a tissue sample by illuminating the slide with at least two beams of light such that the first beam of light illuminates the first side of the slide containing the tissue sample and the second beam of light illuminates the second side of the slide containing the tissue sample, and capturing a first wide field image of a portion of the tissue sample or a whole surface area of the tissue sample from the first side of the slide and a second wide field image of a portion of the tissue sample or a whole surface area of the tissue sample from the second side of the slide using a wide field FLIM for accurate medical diagnosis of a disease condition. Further provided here is a method of evenly illuminating a slide containing a tissue sample in the wide field FLIM by illuminating the slide with a square wave that lasts hundreds of picoseconds to hundreds of microseconds. The resulting fluorescence intensity changes over time at the rising and falling edges of the square wave are subjected to time-domain analysis to yield the fluorescence lifetime and fluorophore intensity.

In various embodiments, the present disclosure relates to a method of evenly illuminating a tissue sample within a wide field light source, comprising positioning a slide containing the tissue sample within the wide field FLIM device such that a focal point of an objective lens of the wide field FLIM device is within the tissue sample contained within the slide, illuminating a top surface of the slide with an excitation light source, illuminating a bottom surface of the slide with the excitation light source, wherein illuminating the top surface of the slide and the bottom surface of the slide with an excitation light source evenly distributes a plurality of excitation photons on a top surface of the tissue sample and a bottom surface of the tissue sample providing an even illumination of the tissue sample, and capturing an image of a portion of the tissue sample from the top surface of the slide and an image of a portion of the tissue sample from the bottom surface of the slide for a medical diagnosis.

In various other embodiments, the present disclosure relates to a method of evenly illuminating a tissue sample within a wide field light source, comprising positioning a slide containing the tissue sample within the wide field FLIM device such that a focal point of an objective lens of the wide field FLIM device is within the tissue sample contained within the slide, illuminating the slide with an excitation light source emitting a square wave, wherein illuminating the slide with a square wave distributes a plurality of excitation photons on the tissue sample, and capturing an image of a portion of or the whole tissue sample from the slide for diagnosis. In many embodiments, the excitation light source may emit a single square wave or a plurality of square waves. In many other embodiments, the slide may be illuminated from a top side only or from a bottom side only or from both the top side and from the bottom side of the slide with an excitation light source emitting a square wave.

In many embodiments, the present disclosure relates to a system of evenly illuminating a tissue sample with a plurality of light beams within a wide field FLIM device, comprising positioning a slide containing the tissue sample within the wide field FLIM device such that a focal point of an objective lens of the wide field FLIM device is within the tissue sample contained within the slide, illuminating a top surface of the slide with a first excitation light, illuminating a bottom surface of the slide with a second excitation light source and capturing an image of a portion of the tissue sample from the top surface of the slide and an image of a portion of the tissue sample from the bottom surface of the slide for a medical diagnosis.

In various embodiments, the top surface and the bottom surface of the slide are illuminated simultaneously, producing simultaneously captured wide field images. In various other embodiments, the top surface and the bottom surface of the slide are illuminated sequentially, resulting in capturing the wide field images captured sequentially, wherein the images can be analyzed individually or the images can be combined for analysis.

Other features will be apparent from the accompanying figures and from the detailed description that follows.

### BRIEF DESCRIPTION OF THE DRAWINGS

The accompanying drawings, where reference numerals refer to embodiments together with the detailed description below, are incorporated in and form part of the specification and serve to further illustrate embodiments of concepts that include the claimed disclosure, and explain various principles and advantages of those embodiments.

The device and embodiments disclosed herein have been represented where appropriate by conventional symbols in the photographs, or drawings, showing only those specific details that are pertinent to understanding the embodiments of the present disclosure so as not to obscure the disclosure with details that will be readily apparent to those of ordinary skill in the art having the benefit of the description herein.

FIG. 1 illustrates a light path of the FLIM illumination system showing, evenly illuminating a slide containing a tissue sample.

FIGS. 2A, 2B and 2C illustrate a decay curve from a single pulse excitation.

FIGS. 3A, 3B and 3C illustrate a decay curve from a square wave excitation.

### DETAILED DESCRIPTION

While the presently disclosed system and method are susceptible of embodiment in many different forms, there are shown in the drawings and will herein be described in detail several specific embodiments with the understanding that the present disclosure is to be considered as an exemplification of the principles of the present technology and is not intended to limit the technology to the embodiments illustrated.

There remain technical challenges to using FLIM technologies for accurate and more reliable diagnosis of medical conditions due to their low fluorescence intensity and poor tissue penetration under certain excitation frequencies. The present invention addresses these problems of shorter excitation signals and in-depth penetration restrictions of the available exciting pulse waveform in wide field FLIM.

In summary, provided here is a method of evenly illuminating a slide containing a tissue sample in a wide field light source by (i) illuminating the slide containing the tissue sample from more than one side or direction, or (ii) illuminating the slide containing the tissue sample using a square wave excitation, or (iii) illuminating the slide containing the tissue sample using a combination of (i) and (ii) and capturing an individual wide field image or a plurality of wide field images of the tissue sample for accurate medical diagnosis of a medical condition.

A single square wave excitation results in enough fluorescence photons to plot an exponential rise of photon count on the rising edge of excitation and the exponential decay of fluorescence at the falling edge of excitation.

Therefore, the current technology is a technical advancement and inventive over prior techniques, as illuminating a tissue sample by a square wave increases the probability that the fluorescence photons would reach larger and deeper tissue areas or cells which is not otherwise possible with the current short excitation pulse techniques within the wide field FLIM. Further, illuminating the tissue sample from more than one side or direction via a wide field light source as compared to illuminating the tissue sample only from a single side, such as from the top as in traditional wide field FLIM devices, exposes more than one surface of the tissue sample to excitation photons and hence capturing images from more than one surface for a better, reliable and accurate medical diagnosis when compared to diagnosis from traditional illumination techniques used in the wide field FLIM.

In various embodiments, the slide containing the tissue sample is placed under the wide field FLIM device, wherein the slide containing the sample is illuminated from a first side of the slide, such as a top side of the slide or from above the slide and a second side of the slide, such as a bottom side of the slide or from below the slide exposing more than one surface area of the tissue sample to fluorescence photons such that a plurality of fluorescence photons reach to a large surface area of the tissue sample providing a better diagnosis of the tissue sample.

The slide containing the tissue sample may be illuminated simultaneously from above and below followed by simultaneous recording of the images, or the slide containing the tissue sample may be illuminated sequentially wherein the slide containing the tissue sample is illuminated first from the first side, followed by illuminating the slide from the second side. When the slide containing the tissue sample is illuminated sequentially, images captured from both sides can be superimposed together for analysis, or the images can be analyzed separately.

Further, a user can control the intensity, frequency, and duration of each beam of light emitting excitation wave, such that the user may illuminate a first side of the slide with a first excitation wave with a certain intensity, frequency and duration and illuminate the second side of the slide with a second excitation wave with intensity, frequency and duration which is not the same as the intensity, frequency, and duration of the first excitation wave.

The user may illuminate the first side and the second side of the slide with a first excitation wave and a second excitation wave, wherein the intensity, frequency, and duration of the first excitation wave and the intensity, frequency, and duration of the second excitation wave are the same. The first excitation wave and the second excitation wave emit a single square wave or a plurality of square waves. The tissue sample may be illuminated by the first excitation wave and the second excitation wave where only one excitation wave from the first excitation wave and the second excitation wave emits a square wave.

The tissue sample is a biological tissue sample or a human tissue sample. The biological tissue sample is a biopsy sample wherein the biopsy sample is not processed for pathological analysis. The tissue sample is a freshly procured biopsy sample.

The slide containing the sample may be illuminated from more than one direction, such as illuminating the slide containing the sample from above and below the slide, or in some embodiments, the sample may be illuminated from more than two directions.

The source of a square wave excitation may be a laser, LED, or other light source commonly used to generate square waves.

The square wave excitation is a low-frequency square-wave or a high-frequency wave excitation producing more fluorescence photons than the fluorescence photons produced by a short excitation pulse. In one aspect, the number of fluorescence photos produced by a single square wave is multiple times more such as hundreds or thousand times more than fluorescence photons produced by a single short pulse wave. Short pulse wave suffers from tissue penetration where the limited number of fluorescence photons from the short pulse wave does not reach beyond a certain depth, such as 100 microns, as compared to illuminating the tissue sample with a square wave where the tissue sample is exposed to plurality of fluorescence photons, wherein fluorescence photons may penetrate beyond the penetration potential of single pulse wave providing a wealth of information of deeper tissue layers and cells.

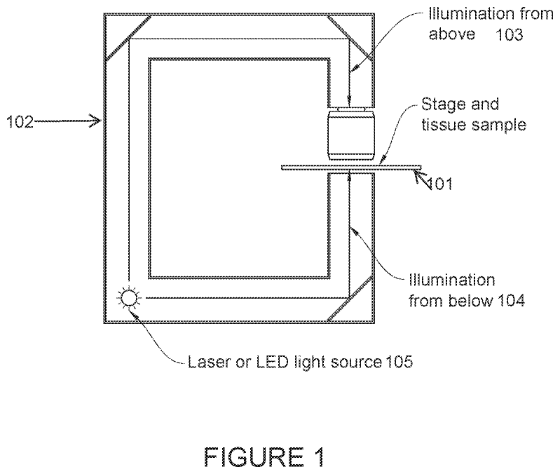

FIG. 1 illustrates a light path of the FLIM illumination system for evenly illuminating a slide containing a tissue sample. As shown in FIG. 1, a slide (101) containing the tissue sample is placed on a stage within the wide field FLIM device (102) such that a focal point of the objective lens of the FLIM device is within the tissue sample contained within the slide such that a first light beam from either LED or a Laser light source (105) falls directly above the tissue sample or illuminates the tissue sample from directly above (103) or on the top surface of the tissue sample present on the slide and a second beam of light falls directly below the tissue sample or illuminates the tissue sample from below (104) or on the bottom side or surface of the slide so the light falls directly on the bottom surface of the tissue sample. Illuminating a biological tissue sample from more than one side by a large number of fluorescence photons as compared to the number of fluorescence photons from a short pulse followed by capturing wide field images from a plurality of surface areas of the biological sample prevents false positives and negatives and provides an accurate medical diagnosis of a disease condition as compared to traditional FLIM technologies using a single photon excitation.

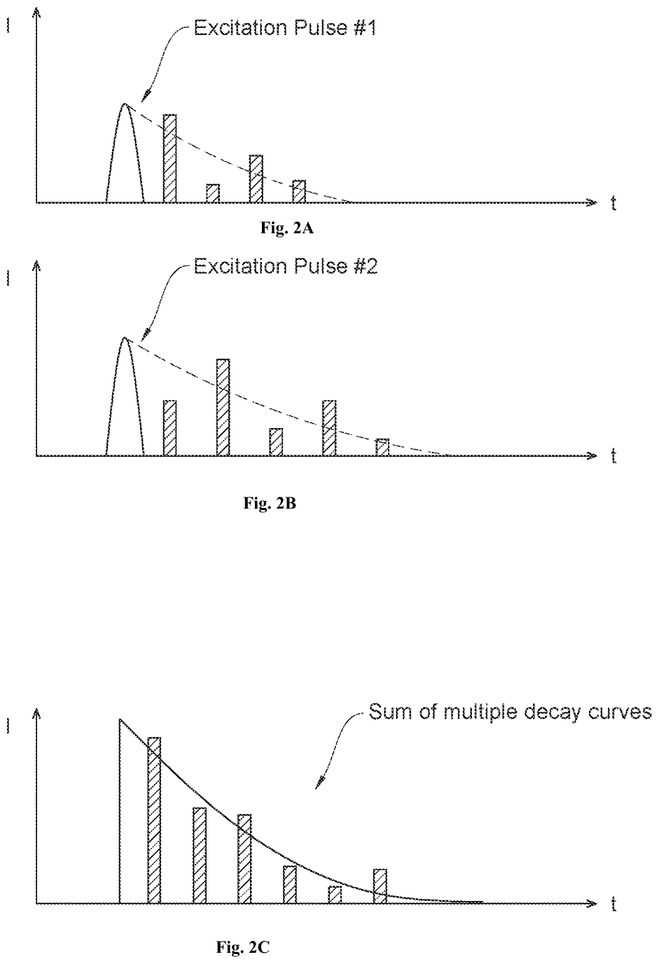

FIGS. 2A, 2B and 2C illustrate a decay curve from a single pulse excitation. As shown in FIGS. 2A and 2B, a sample illuminated with only a single excitation pulse (excitation pulse #1 and excitation pulse #2 respectively) at a given time shows a rapid decay curve generated due to spontaneous emission of the photon and hence provides a choppy decay curve. In order to create a smooth decay curve for analysis, a user would have to sum up more than one decay curve, as shown in FIG. 2C for analysis. Thus, using a single excitation pulse technology, the tissue sample would be illuminated by a series of short excitation pulses and the decay curves are summed up to create a smooth decay curve for analysis.

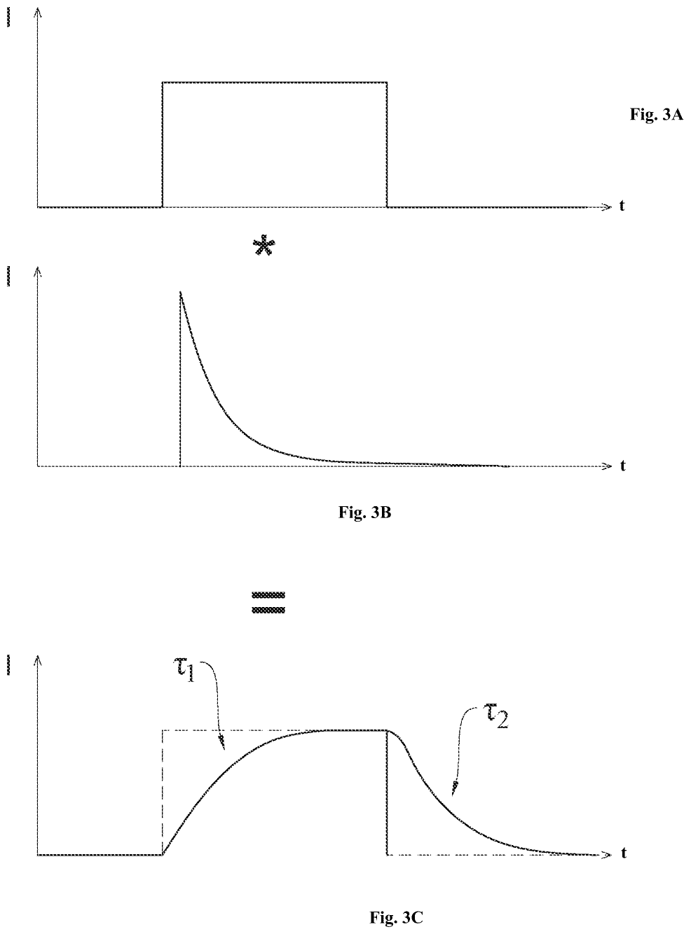

FIGS. 3A, 3B and 3C illustrate a decay curve from a square wave excitation. FIG. 3A shows a single square wave excitation to illuminate a tissue sample, wherein the square wave provides a wider wave area producing a plurality of fluorescence photons in comparison to the short pulse excitation at a given time. Further, as shown in FIG. 3B, once the plurality of photons is excited, the intensity of the fluorescence decays exponentially. The resulting fluorescence intensity changes over time (τ1 and τ2), which can be calculated as fluorescence lifetimes (τ1−τ2−τ), as shown in FIG. 3C.

In the description, for purposes of explanation and not limitation, specific details are set forth, such as particular embodiments, procedures, techniques, etc. in order to provide a thorough understanding of the present technology. However, it will be apparent to one skilled in the art that the present technology may be practiced in other embodiments that depart from these specific details.

While specific embodiments of, and examples for, the device and process are described above for illustrative purposes, various equivalent modifications are possible within the scope of the system, as those skilled in the relevant art will recognize. For example, while devices or processes are presented in a given order, alternative embodiments may perform routines having steps in a different order, and some processes or steps may be deleted, moved, added, subdivided, combined, and/or modified to provide alternative or sub-combinations. Each of these processes may be implemented in a variety of different ways. Also, while processes or steps are shown as being performed in series, these processes or steps may instead be performed in parallel or at other times.

While various embodiments have been described above, it should be understood that they have been presented by way of example only and not in limitation. The descriptions are not intended to limit the scope of the present technology to the particular forms set forth herein. On the contrary, the present descriptions are intended to cover such alternatives, modifications, and equivalents as may be included within the spirit and scope of the present technology as appreciated by one of ordinary skill in the art. Thus, the breadth and scope of a preferred embodiment should not be limited by any of the above-described exemplary embodiments.

What is claimed is:

1. A method of evenly illuminating a tissue sample within a wide field Fluorescence Lifetime Imaging Microscopy (FLIM) device, comprising:
positioning a slide containing the tissue sample within the wide field FLIM device such that a focal point of an objective lens of the wide field FLIM device is within the tissue sample contained within the slide;
illuminating a top surface of the slide with an excitation light source;
illuminating a bottom surface of the slide with the excitation light source; and
capturing an image of a portion of the tissue sample from the top surface of the slide and an image of a portion of the tissue sample from the bottom surface of the slide for diagnosis.

2. A method of evenly illuminating a tissue sample within a wide Fluorescence Lifetime Imaging Microscopy (FLIM) device, comprising:
positioning a slide containing the tissue sample within the wide field FLIM device such that a focal point of an objective lens of the wide field FLIM device is within the tissue sample contained within the slide,
illuminating either a top surface of the slide with an excitation light source emitting a square wave or a bottom surface of the slide with the excitation light source emitting the square wave; and
capturing an image of a portion of the tissue sample from the top surface of the slide or an image of a portion of the tissue sample from the bottom surface of the slide for diagnosis.
